(19) United States
(12) Patent Application Publication
(10) Pub. No.: US 2026/0027231 A1
(43) Pub. Date: Jan. 29, 2026

(54) GENOME EDITING OF THE KOZAK SEQUENCE FOR TREATING DISEASES

(71) Applicant: UNIVERSITA' DEGLI STUDI DI TRENTO, Trento (IT)

(72) Inventors: Alessandro QUATTRONE, Trento (IT); Chiara AMBROSINI, Trento (IT); Anna CERESETO, Trento (IT); Gianluca PETRIS, Trento (IT); Eliana DESTEFANIS, Trento (IT); Alberto RAOSS, Trento (IT)

(21) Appl. No.: 18/996,418

(22) PCT Filed: Aug. 4, 2023
(86) PCT No.: PCT/IT2023/050194

§ 371 Date: Jan. 17, 2025

(30) Foreign Application Priority Data
Aug. 5, 2022 (IT) 102022000016884

(51) Int. Cl. A61K 48/00; A61K 31/7088; A61P 43/00; C12N 9/22; C12N 15/113; C12N 15/67

(52) U.S. Cl. CPC A61K 48/005; A61K 31/7088; A61P 43/00; C12N 9/222; C12N 15/113; C12N 15/67; C12N 2310/20

(57) ABSTRACT

The present invention relates to the medical field of single-gene disorders caused by functional loss or gain of an allele. The innovative approach developed being based on editing the human genome at the level of the Kozak sequence by means of CRISPR-Cas programmable nucleases. Particularly, the present invention relates to variant Kozak sequences and related in vitro or in vivo methods for obtaining such variant Kozak sequences for therapeutic applications in the treatment of single-gene diseases caused by monoallelic losses or gains. These in vitro and in vivo methods include CRISPR-Cas homology-directed repair, CRISPR-Cas prime editing, CRISPR-Cas base editing or genome editing with other programmable RNA-guided nucleases, and the introduction of specific nucleotide conversions in the Kozak sequence of genes causative of diseases. These nucleotide conversions enhance or inhibit the translation of the mRNA produced by the gene, compensating for the functional loss or gain of one allele in the diseases.

### TECHNICAL FIELD

The present invention relates to the medical field of single-gene disorders caused by functional loss or gain of an allele. The innovative approach developed being based on editing the human genome at the level of the Kozak sequence by means of CRISPR-Cas programmable nucleases. Particularly, the present invention relates to variant Kozak sequences and related in vitro or in vivo methods for obtaining such variant Kozak sequences for therapeutic applications in the treatment of single-gene diseases caused by monoallelic losses or gains. These in vitro and in vivo methods include CRISPR-Cas homology-directed repair, CRISPR-Cas prime editing, CRISPR-Cas base editing or genome editing with other programmable RNA-guided nucleases, and the introduction of specific nucleotide conversions in the Kozak sequence of genes causative of diseases. These nucleotide conversions enhance or inhibit the translation of the mRNA produced by the gene, compensating for the functional loss or gain of one allele in the diseases.

### STATE OF THE ART

Disorders caused by functional loss or gain of a single allele are hundreds. Particularly, haploinsufficiency (HI) is a genetic condition by which mutational inactivation of a single allele leads to reduced protein levels and is enough to produce the disease phenotype. More than 300 human conditions, ranging from cancer predisposition to developmental and neurological single-gene disorders, are caused by haploinsufficiency. Gene duplication (GD) is instead a gain of a single allele leading to increased protein levels and producing a disease phenotype. Dozens of human conditions are caused by gene duplication.

The Kozak consensus sequence was defined for the first time in the 1980s as the optimal nucleotide context around the starting codon of a protein. It is now widely accepted that the Kozak sequence plays a fundamental role in the regulation of translation, by contracting specific interactions into the 48S initiation complex.

The impact of the Kozak sequence on disease is exemplified by hereditary and sporadic diseases in which translation efficiency is affected because of point mutations and allelic variations, respectively, near the AUG starting codon. For instance, a C-to-T mutation within the Kozak sequence, in position −1 with respect to the AUG codon (c-1.C>T), of the α-tocopherol transfer protein gene reduces protein levels and causes the AVED (ataxia with vitamin E deficiency) monogenic disorder. As another example, a T/C polymorphism in the −1 position of the CD40 gene (−1T>C, rs1883832) is associated with increased CD40 translation, therefore predisposing to Graves' disease and coronary heart disease.

Despite the apparently strict pattern of the motif describing the Kozak consensus sequence, sequence variability is present around the AUG starting codon in the human genome and in the genomes of other vertebrates, and more recent efforts aimed at measuring the strength of different AUG starting codons as a function of the surrounding bases have found substantial variance. These results open the possibility that several genes are translationally controlled by suboptimal Kozak sequences.

Given the cited data and its major role in regulating translation, the Kozak sequence is also a potential target for modulating gene expression.

### SUMMARY OF THE INVENTION

The Applicant has now found a genome editing approach aimed at rescuing diseases caused by functional loss or gain of a single allele. Such an approach can exploit any genome editing platform capable of introducing single or multiple base conversions in a short stretch of DNA. Therefore, CRISPR-Cas homology-directed repair, CRISPR-Cas prime editing, CRISPR-Cas base editing or any other programmable RNA-guided nuclease fused to effector proteins allowing for the instalment of base conversions into the genome are suitable.

In one of the embodiments of the invention, the Applicant has focused on the modification of the Kozak sequence to enhance the translational efficiency of the wild-type allele with reference to HI genes of interest. In other words, in this embodiment of the invention, the procedure upregulates protein production through variation of the Kozak sequence, with therapeutic purposes.

Particularly, as an example of the method, the Applicant has now found specific nucleotide conversions in the Kozak sequences of selected HI genes to be used in the treatment of HI diseases. These diseases belong to the classes of developmental disabilities, metabolic syndromes, eye disorders, and hematopoietic diseases.

In another embodiment of the invention, the Applicant has focused on the modification of the Kozak sequence to repress the translational efficiency of the three alleles with reference to GD genes of interest. In other words, in this embodiment of the invention, the procedure downregulates protein production through variation of the Kozak sequence, with therapeutic purposes.

Therefore, a first object of the present invention relates to a nucleotide sequence according to claim 1.

Advantageously, the invention allows the use of Kozak sequences targeted by genome editing approaches aimed at modulating translational efficiency, manipulating it with the aim of upregulating or downregulating protein production with therapeutic purposes. Other advantages of the approach on which the invention is based are the following. Firstly, genome editing methods can install permanent nucleotide conversions in the genomic DNA; secondly, nucleotide conversions on the Kozak sequence allow to target HI genes by inducing small, controlled increases in translational efficiency and this is crucial because the final goal is to achieve a gain in protein levels sufficient to rescue the HI phenotype but not high enough to create imbalances and be incompatible with cell physiology; third, the action on a cis sequence universally controlling translation produces the desired effect on any cell type of the body, irrespective of gene expression variability, and dampens noise in the system; fourth, several different Kozak variants can be tested for each gene, assuring flexibility; last, any mutated HI gene causing disease can in principle be rescued in any patient because the approach does not depend on the correction of a specific disease.

Further objects, characteristics, preferred embodiments and advantages of the invention are reported below. The scope of the invention being defined in the attached claims.

### BRIEF DESCRIPTION OF THE DRAWINGS

FIG. 1 shows the boosting of a suboptimal Kozak sequence by base editing. A. Sanger sequencing chromatograms representing the wild-type (EGFP-1C) and the mutated EGFP version (EGFP-1T), with a single variation in position-1 of the Kozak sequence. B. Western blot analysis of EGFP and mCherry expression in HEK293T cells transiently transfected with EGFP-1C or EGFP-1T plasmids. C. Representative FACS dot plots of HEK293T cells 3 days after transient transfection. D. FACS analysis of HEK293T cells transiently transfected with the respective plasmids. The data are normalised over EGFP-1C and are reported as mean±SD of n=3 biological replicates. Statistically significant differences were calculated by unpaired t-test. E. Representative Sanger sequencing chromatograms of HEK293T cells edited with the ABE7.10 base editor and sg-1, compared with ABE7.10 combined with a scrambled sgRNA (sgCTRL). F. Percentage of correct T-to-C conversion analysed with the EditR software. G. Western blot analysis of EGFP and mCherry expression in HEK293T cells edited with ABE 7.10 or ABEmax combined with sg-1 or sgCTRL. H. Representative FACS dot plots of cells edited with ABE7.10 and sg-1, compared with ABE7.10 combined with a scrambled sgRNA (sgCTRL) 3 days after transfection. I. FACS analysis of EGFP expression in cells transfected with the base editors (ABE7.10 and ABEmax) and sgCTRL or sg-1. Data are means±SD from n=3 biological replicates. Statistically significant differences were calculated by unpaired t-test (p value=0.0483)

FIG. 2 refers to the high-throughput determination of protein levels from Kozak sequence variants. A. mCherry expression of the transduced cells in FACS-seq first round of sorting. 5×10₆ mCherry-positive cells (23.1% of the total) were sorted. B. FACS-seq second round of sorting. C. mCherry-positive cells from the gate drawn in B. were divided into 4 gates according to EGFP/mCherry expression, defined in such a way that each bin contains 25% of the total population of interest. D. The heatmap represents the distribution of the candidate HI genes and variants which passed the statistical analysis. In the upper panel, the Kozak variants are represented. The WT Kozak sequences of the HI genes are shown in the lower panel. Each column corresponds to one of the four gates, while each row stands for one of the Kozak variants. E. Logo representation of the Kozak sequences extracted from each of the four gates. In each panel, the positions along the Kozak sequence (with A of ATG being position +1) are represented on the x-axis, and the probability of occurrence of each base is shown on the y-axis. Gate 1 (upper panel) represents the lowest translational efficiency, while gate 4 (lower panel) corresponds to the most performing Kozak sequences. Relevant positions (−3 and +5) are highlighted in yellow. F. Percentage of the count per million reads (CPM) in the 4 gates of the wild-type (WT) and the respective variants (Var) of the 5 selected genes.

FIG. 3 refers to the Validation of actionable hit variants. A. Wild-type (WT) and variants (Var) Kozak sequences of the selected hit genes. B. Translational enhancement analysed as EGFP/mCherry expression by high content image analysis. The violin plots report the data distribution from n=3 biological replicates. The dashed line indicates the population median. C. The histogram represents the mean of the populations analysed by high content image analysis. Data are means±SD from n=3 biological replicates. The numbers indicate the percentage of mean increase of the variants over the WT. Statistically significant differences were calculated using the unpaired t-test of each variant versus the corresponding WT.

FIG. 4 refers to the validation of actionable hit variants for PMP22 translational repression. A. Wild-type (WT) and variants (Var) Kozak sequences of the PMP22 gene. B. Translational repression analysed as EGFP/mCherry expression by high content image analysis. The violin plots report the data distribution from n=2,3 biological replicates. The dashed line indicates the population median. C. The histogram represents the mean of the populations analysed by high content image analysis. Data are means±SD from n=2,3 biological replicates. Statistically significant differences were calculated using the unpaired t-test of each variant versus the WT.

FIG. 5 refers to the base editing of NCF1 to replicate the desired variants. A. Schematic representation of the NCF1 wild-type (WT), variant 2 (Var 2), and variant 4 (Var 4) Kozak sequences. The starting codon is bold blue; the base changes in the variants are highlighted in pink. B. Editing efficiency in the Raji bulk population at target and bystander (in red) guanines analysed with the EditR software 5 days post-electroporation of AncBE4max and sgNCF1 or sgCTRL. The percentage of corrected G-to-A conversions (y-axis) is shown for each position in the NCF1 Kozak sequence (x-axis, with the A of ATG being position +1). Data are means±SD from n=3 independent experiments. C. Editing efficiency in the two clones isolated from the bulk population (Var 2 and Var 4 cells) at target and bystander (in red) guanines. D. Sanger sequencing chromatograms of NCF1 Kozak sequence in Raji WT, Var 2, and Var 4cells. E. Western blot analysis of the p47ₚₕₒₓ protein in Raji cells (WT, Var 2, and Var 4). One representative blot result is shown. The arrow indicates the 47 KDa band corresponding to p47ₚₕₒₓ. F. Western blot quantification. p47ₚₕₒₓ levels were normalised on the housekeeping protein, and the fold change with respect to the WT levels is shown, n=3 biological replicates. G. qPCR of NCF1 on WT, Var 2, or Var 4 Raji cells. Data are means±SD from n=3 independent experiments. H. Representative western blot of two polysomal markers (RPS6 and RPL26) in the fractions isolated by sucrose gradient centrifugation. The input is the cellular cytoplasmic lysate loaded on the sucrose gradient. tot=fractions corresponding to the total RNA; pol-fractions selected as polysomes and used in I. I. Translational efficiency (TE) quantification of NCF1 in Var 2 and Var 4 cells with respect to the WT cells. TE is the ratio between polysomal (fractions 8-9) and total (fractions 4-9) mRNA levels (fold change polysome/fold change total) measured by qPCR. Data are means±SD from n=3 independent experiments. Statistically significant differences were calculated by unpaired t-test of each variant versus the WT.

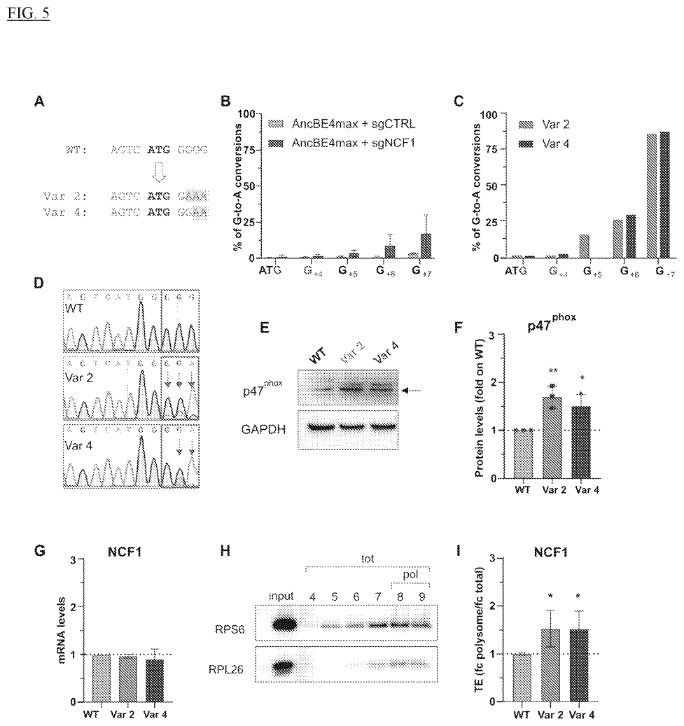

### DETAILED DESCRIPTION OF THE INVENTION

For the scope of the invention, some terms and expressions, used in the present description and in the attached claims, are provided below.

As intended herein, the term “CRISPR” refers to Clustered Regularly Interspaced Short Palindromic Repeat systems or loci, a bacterial adaptive immune system that protects against invading mobile genetic elements. The term “Cas” (CRISPR-associated protein) refers to an RNA-guided programmable endonuclease, that recognizes a protospacer-adjacent motif (PAM sequence) and cleaves the invading nucleic acid in a region complementary to the sequence encoded by the spacer, encoded in the CRISPR array. The term “Cas9n” refers to a partially inactive Cas9 endonuclease. Reference: Jinek, M., Chylinski, K., Fonfara, I., Hauer, M., Doudna, J. A., & Charpentier, E. (2012). “A programmable dual-RNA-guided DNA endonuclease in adaptive bacterial immunity”. Science, 337(6096), 816-821.

As intended herein, the expression “base editor” refers to the fusion of a deaminase to a Cas9n, in a tool able to perform single-base conversions. There are three types of base editor: cytosine base editors (CBE), able to convert C-G into T-A base pair, and adenine base editors (ABE), able to perform the substitution from A-T to G-C base pair, and adenine to cytosine base editors, able to perform the substitution from A-T to C-G. Reference: Komor, A. C., Kim, Y. B., Packer, M. S., Zuris, J. A., & Liu, D. R. (2016). “Programmable editing of a target base in genomic DNA without double-stranded DNA cleavage”. Nature, 533 (7603), 420-424. Chen L, Hong M, Luan C, Gao H, Ru G, Guo X, Zhang D, Zhang S, Li C, Wu J, Randolph P B, Sousa A A, Qu C, Zhu Y, Guan Y, Wang L, Liu M, Feng B, Song G, Liu D R, Li D., Nat Biotechnol. 2023 Jun. 15.

As intended herein, the term “gRNA” (single-guide RNA) refers to an RNA molecule that functions as a guide to direct the Cas9 protein or base editor or prime editor to the locus of interest, complementary to its spacer sequence and adjacent to the PAM motif that is within 30 nucleotides to the ATG starting codon, either upstream or downstream.

As intended herein, the term “haploinsufficiency” refers to a molecular mechanism in which the mutational inactivation of one allele of a gene is sufficient to produce the disease phenotype. “Haploinsufficient diseases” are disorders caused by such a process, characterized by insufficient quantities of a particular protein. The term “gene duplication disease” refers instead to a molecular mechanism in which the duplication of a gene usually results in an increase in the quantity of a particular protein, which cause disease.

As intended herein, the term “homology-directed repair” (HDR) refers to a repair pathway induced by a double-strand break in the DNA. As opposed to non-homologous-end joining repair, HDR allows inserting a precise edit in the DNA, by providing a donor DNA molecule encoding for the desired edit, that will be used as a template for DNA repair. In genome editing applications, HDR is exploited to insert genetic information encoded by the donor DNA after Cas9 cleavage of the target locus. Reference: Doudna, J. A., & Charpentier, E. (2014). “The new frontier of genome engineering with CRISPR-Cas9”. Science, 346(6213), 1258096.

As intended herein, the expression “Kozak consensus sequence” or “Kozak sequence” refers to a DNA sequence motif that functions as the translation initiation site in most eukaryotic mRNAs. It ensures that translation starts at the correct site on the mRNA, mediating the reading of the AUG initiation codon in the ribosomes.

As intended herein, the expression “Kozak variant sequence” or “Kozak variant” refers to an alternative Kozak sequence designed by substitution of some of the 4 nucleotides flanking on both sides of the ATG codon of a wild-type Kozak sequence, having the ATG codon kept constant (i.e., NNNN ATG NNNN). One variant can contain multiple conversions belonging to the same type.

As intended herein, the expression “prime editor” refers to a complex characterized by a Cas9n fused to a reverse transcriptase enzyme. This complex is able to write new genetic information at a target locus thanks to the programmable pegRNA (prime editing guide RNA), which codes both for the target site and the template with the edit that needs to be inserted. Reference: Anzalone, A. V., Randolph, P. B., Davis, J. R., Sousa, A. A., Koblan, L. W., Levy, J. M., . . . & Liu, D. R. (2019). “Search-and-replace genome editing without double-strand breaks or donor DNA.” Nature, 576(7785), 149-157.

As intended herein, the expressions “translational modulator”, “translational enhancer”, or “translational repressor”, are cis sequences and trans factors endowed with the ability to modulate, enhance, or repress, respectively, the translational efficiency of a given mRNA. More specifically in our context, the expression refers to variant Kozak sequences endowed with the same ability.

As intended herein, the term “vector” refers to a nucleic acid that is able to enter into a host cell, mutate and replicate within the host cell, and then transfer a replicated form of the vector into another host cell.

Advantageously, in one of the embodiments of the present invention, in order to mutate the Kozak sequence base editors are used. Moreover, in one of the embodiments of the present invention, the disease genes targeted are HI disease genes. In such cases, the change of one or few nucleotides in the Kozak sequence has to be compatible with the action of base editors and has to allow increasing the amount of the encoded protein, thus compensating for the deleterious effects of the functional loss of one allele. In order to do so, in the present invention gRNAs are appropriately selected in such a way as to induce the base editor to modify one of a few nucleotides inducing translational enhancement with respect to the wild-type Kozak sequence. The invention, therefore, relates to a Kozak variant nucleotide sequence selected from SEQ ID NO:1 to SEQ ID NO:58 for use in the treatment of a HI disease.

According to a preferred embodiment, the HI disease is selected from the following disease areas: developmental disabilities, metabolic syndromes, eye disorders, and hematopoietic diseases.

For each above-mentioned HI disease area, below the corresponding specific sequences are listed, as well as further related details.

According to a preferred embodiment, when the HI disease belongs to the class of the developmental disabilities, the nucleotide sequence is selected from: SEQ ID NO:1 to SEQ ID NO:28. The HI disease genes causing the developmental disability when mutated are selected from: DYRK1A, GRIN2B, NRXN1, and STXBP1. The developmental disability disease is selected from: intellectual developmental disorder, autosomal dominant 7 (OMIM #614104), caused by functional loss of an allele of the DYRK1A gene; intellectual developmental disorder, autosomal dominant 6, with or without seizures (OMIM #613970), caused by functional loss of an allele of the GRIN2B gene; chromosome 2p16.3 deletion syndrome (OMIM #614332), caused by functional loss of an allele of the NRXN1 gene; developmental and epileptic encephalopathy 4 (OMIM #612164), caused by functional loss of an allele of the STXBP1 gene.

When the developmental disability is intellectual developmental disorder, autosomal dominant 7, the nucleotide sequence is selected from SEQ ID NO:1 to SEQ ID NO:3. When the developmental disability is intellectual developmental disorder, autosomal dominant 6, with or without seizures, the nucleotide sequence is selected from: SEQ ID NO:4 to SEQ ID NO:8. When the developmental disability is chromosome 2p16.3 deletion syndrome, the nucleotide sequence is selected from: SEQ ID NO:9 to SEQ ID NO:14. When the developmental disability is developmental and epileptic encephalopathy 4, the nucleotide sequence is selected from: SEQ ID NO:15 to SEQ ID NO:28.

According to another preferred embodiment, when the HI disease is of the class of metabolic syndromes, the nucleotide sequence is selected from: SEQ ID NO:29 to SEQ ID NO:41. The HI disease genes causing the metabolic syndromes are selected from: HNF1A, GHRL, and PROX1. The metabolic syndrome is selected from: maturity-onset diabetes of the young, type 3 (OMIM #600496), caused by functional loss of an allele of the HNF1A gene; susceptibility to obesity (OMIM #601665), caused by functional loss of an allele of the GHRL gene; adult-onset obesity and lymphatic vascular disease, caused by functional loss of an allele of the PROX1 gene.

When the metabolic syndrome disease is maturity-onset diabetes of the young, type 3, the nucleotide sequence is selected from: SEQ ID NO:29 to SEQ ID NO:33. When the metabolic syndrome is susceptibility to obesity, the nucleotide sequence is selected from: SEQ ID NO:34 to SEQ ID NO:38. When the metabolic syndrome is adult-onset obesity and lymphatic vascular disease, the nucleotide sequence is selected from: SEQ ID NO:39 to SEQ ID NO:41.

According to another preferred embodiment, when the HI disease is of the class of eye disorders, the nucleotide sequence is selected from: SEQ ID NO:42 to SEQ ID NO:53. The HI disease genes causing the eye disorder are selected from: EYA1, OPA1, COL2A1. The eye disorder is selected from: branchiootorenal syndrome 1 (OMIM #113650), caused by functional loss of an allele of the EYA1 gene; optic atrophy 1 (OMIM #165500), caused by functional loss of an allele of the OPA1 gene; Stickler syndrome type 1, nonsyndromic ocular (OMIM #609508), caused by functional loss of an allele of the COL2A1 gene.

When the eye disorder is branchiootorenal syndrome 1, the nucleotide sequence is selected from: SEQ ID NO:42 or SEQ ID NO:43. When the eye disorder is optic atrophy 1, the nucleotide sequence is selected from: SEQ ID NO:44 to SEQ ID NO:50. When the eye disorder is Stickler syndrome type 1, nonsyndromic ocular, the nucleotide sequence is selected from: SEQ ID NO:51 to SEQ ID NO:53.

According to another preferred embodiment, when the HI disease is a hematopoietic disease, the nucleotide sequence is selected from: SEQ ID NO:54 to SEQ ID NO:58. The HI gene involved in the hematopoietic disease is NCF1. The hematopoietic disease is chronic granulomatous disease (OMIM #233700).

In an other embodiment of the present invention, the disease genes targeted are GD disease genes. In such cases, the change of one or few nucleotides in the Kozak sequence has to be compatible with the action of base editors and has to allow decreasing the amount of the encoded protein, thus compensating for the deleterious effects of the functional gain of one allele. In order to do so, in the present invention gRNAs are appropriately selected in such a way as to induce the base editor to modify one of a few nucleotides and obtain translational repression with respect to the wild-type Kozak sequence. The invention, therefore, relates to a Kozak variant nucleotide sequence selected from SEQ ID NO:61 to SEQ ID NO:65 for use in the treatment of a HI disease.

According to a preferred embodiment, the HI disease is selected from the following disease areas: developmental disabilities, metabolic syndromes, eye disorders, and hematopoietic diseases.

For each above-mentioned HI disease area, below the corresponding specific sequences are listed, as well as further related details.

According to a preferred embodiment, when the HI disease belongs to the class of developmental disabilities, the nucleotide sequence is selected from: SEQ ID NO:1 to SEQ ID NO:28. The HI disease genes causing the developmental disability when mutated are selected from: DYRK1A, GRIN2B, NRXN1, and STXBP1. The developmental disability disease is selected from: intellectual developmental disorder, autosomal dominant 7 (OMIM #614104), caused by functional loss of an allele of the DYRK1A gene; intellectual developmental disorder, autosomal dominant 6, with or without seizures (OMIM #613970), caused by functional loss of an allele of the GRIN2B gene; chromosome 2p16.3 deletion syndrome (OMIM #614332), caused by functional loss of an allele of the NRXN1 gene; developmental and epileptic encephalopathy 4 (OMIM #612164), caused by functional loss of an allele of the STXBP1 gene.

Furthermore, the invention also relates to a vector suitable for genome editing comprising any gRNA designed to edit the wild-type Kozak sequence in order to obtain any of the variant Kozak nucleotide sequence selected from the group SEQ ID NO:1-SEQ ID NO:58. These gRNAs are such that their targeting sequence corresponds to a target domain adjacent to a PAM sequence that is within 30 nucleotides to the ATG starting codon embedded in the Kozak sequence, either upstream or downstream

According to a preferred embodiment, when the disease is chronic granulomatous disease, the gRNA editing the wild-type Kozak sequence of NCF1 is encoded by SEQ ID NO:59.

According to a preferred embodiment, when the disease is chronic optic atrophy, the gRNA editing the wild-type Kozak sequence of OPA1 is encoded by SEQ ID NO:60.

Furthermore, the invention also relates to a pharmaceutical composition containing at least one gRNA designed to obtain any one of the variant Kozak sequences selected from SEQ ID NO:1 to SEQ ID NO:58, a suitable vector for genome editing and a genome editing complex selected between those necessary for the methods of CRISPR-Cas homology-directed repair, CRISPR-Cas prime editing, CRISPR-Cas base editing or any other method based on programmable RNA-guided nucleases fused to effector proteins allowing for the introduction of one or more nucleotide conversions and at least one pharmaceutically acceptable excipient. According to a preferred embodiment this pharmaceutical composition is intravenously injectable.

Thus, an object of the present invention is a variant Kozak nucleotide sequence obtained by genome editing methods acting as translational modulator of a protein-encoding gene in the treatment of a disease wherein the expression of said protein is altered, wherein said variant Kozak nucleotide sequence replaces, in vitro or in vivo, the wild-type Kozak sequence.

Preferably, an object of the present invention is a variant Kozak nucleotide sequences according to claim 1, wherein said genome editing methods are selected from the group consisting of CRISPR-Cas homology-directed repair, CRISPR-Cas prime editing, CRISPR-Cas base editing or any other method based on programmable RNA-guided nuclease fused to effector proteins allowing for the introduction of one or more nucleotide conversions.

According to an embodiment, is preferred a variant Kozak nucleotide sequence as said above wherein said translation modulator is a translation enhancer or a translation repressor.

According to an embodiment, is preferred a variant Kozak nucleotide sequence said above selected from the group consisting of SEQ ID NO:1-SEQ ID NO:58 as translation enhancers wherein said variant Kozak nucleotide sequences replace, in vitro or in vivo, the wild-type Kozak sequence.

An other object is the use of a variant Kozak nucleotide sequence said above for increasing the translational efficiency of a protein-encoding gene in the treatment of haploinsufficiency diseases.

According to a preferred embodiment is preferred the use of a variant Kozak nucleotide sequence said above wherein said haploinsufficiency disease is selected from the following disease classes: developmental disabilities, metabolic syndromes, eye disorders and hematopoietic diseases.

According to an embodiment, is preferred the use of a variant Kozak nucleotide sequence said above, wherein the developmental disability is selected from: intellectual developmental disorder, autosomal dominant 7; intellectual developmental disorder 6, with or without seizures; 2p16.3 deletion syndrome; developmental and epileptic encephalopathy 4.

According to an embodiment, is preferred the use of a variant Kozak nucleotide sequence according to any one of claims 6-7, wherein:
when the developmental disability is intellectual developmental disorder, autosomal dominant 7, the nucleotide sequence is selected from SEQ ID NO:1 to SEQ ID NO:3;
when the developmental disability is intellectual developmental disorder, autosomal dominant 6, with or without seizures, the nucleotide sequence is selected from SEQ ID NO:4 to SEQ ID NO:8;
when the developmental disability is chromosome 2p16.3 deletion syndrome, the nucleotide sequence is selected from SEQ ID NO:9 to SEQ ID NO:14;
when the developmental disability is developmental and epileptic encephalopathy 4, the nucleotide sequence is selected from SEQ ID NO:15 to SEQ ID NO:28;

According to an embodiment, is preferred the use of a variant Kozak nucleotide sequence said above, wherein the metabolic syndrome is selected from: maturity-onset diabetes of the young, type 3; susceptibility to obesity; lymphatic vascular defects and/or adult-onset obesity.

According to an embodiment, is preferred the use of a variant Kozak nucleotide sequence said above, wherein:
when the metabolic syndrome is maturity-onset diabetes of the young, type 3, the nucleotide sequence is selected from SEQ ID NO:29 to SEQ ID NO:33;
when the metabolic syndrome is susceptibility to obesity, the nucleotide sequence is selected from SEQ ID NO; 34 to SEQ ID NO:38;
when the metabolic syndrome is lymphatic vascular defects and/or adult-onset obesity, the nucleotide sequence is selected from SEQ ID NO:39 to SEQ ID NO:41.

According to an embodiment, is preferred the use of a variant Kozak nucleotide sequence said above, wherein the eye disorder is selected from branchiootorenal syndrome, optic atrophy, Stickler syndrome type 1, nonsyndromic ocular.

The use of a variant Kozak nucleotide sequence said above, wherein:
when the eye disorder is branchiootorenal syndrome, the nucleotide sequence is selected from SEQ ID NO:42 or SEQ ID NO:43;
when the eye disorder is optic atrophy, the nucleotide sequence is selected from SEQ ID NO:44 to SEQ ID NO:50;
when the eye disorder is Stickler syndrome type 1, nonsyndromic ocular, the nucleotide sequence is selected from SEQ ID NO:51 to SEQ ID NO:53.

According to an embodiment, is preferred the use of a variant Kozak nucleotide sequence said above wherein when the hematopoietic disease is chronic granulomatous disease, said nucleotide sequence is selected from SEQ ID NO:54 to SEQ ID NO:58.

An other object is a gRNA designed to edit the wild-type Kozak sequence in order to obtain any one of the variant Kozak nucleotide sequences selected from the group SEQ ID NO:1-SEQ ID NO:58, characterized in that its targeting sequence corresponds to a target domain adjacent to a PAM sequence that is within 30 nucleotides to the ATG starting codon, either upstream or downstream.

According to an embodiment, is preferred a gRNA sad above editing the wild-type Kozak sequence of NCF1 wherein the gRNA has the nucleotide sequence SEQ ID NO:59.

According to an other embodiment, is preferred a gRNA said above editing the wild-type Kozak sequence of OPA1 wherein the gRNA has the nucleotide sequence SEQ ID NO:60.

An other object is a vector for genome editing comprising any one of the gRNAs said above

An other object is a pharmaceutical composition comprising the vector said above, together with other suitable components for in vivo genome editing.

According to an embodiment is preferred a pharmaceutical composition said above, which is intravenously injectable.

### EXAMPLES

### Experimental Part

### Base Editing-Mediated Kozak Optimization Enhances Translation in a Reporter System

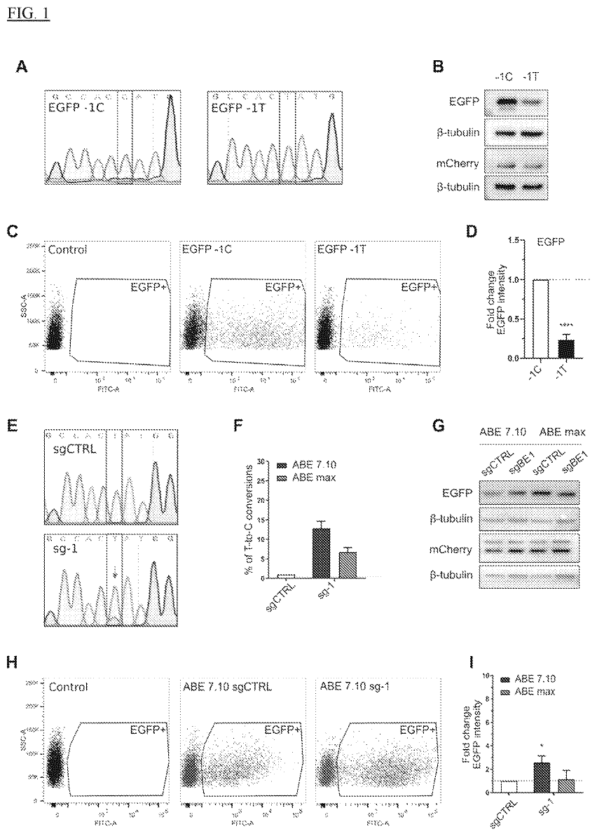

In order to demonstrate the feasibility of the proposed approach, an experiment focused on enhancing EGFP translation from a reporter vector was performed. We created two versions of the bicistronic reporter vector pWPT-EGFP-IRESmCherry: EGFP wild-type Kozak sequence (C in position −1, EGFP-1C) or a suboptimal motif having a T in position −1 (EGFP-1T) (FIG. 1A). This single base change reduced EGFP translation by 4-5 fold (FIG. 1B, C, D). Secondly, we corrected this nucleotide variation with base editors and observed a significant increase in EGFP expression (FIG. 1E-I). These data confirm that base editors can be used to selectively mutate single nucleotides in the Kozak sequence.

### Design and Generation of the Library of Actionable Kozak Variants

We screened wild-type (WT) Kozak sequences of annotated HI genes and compared them with respective variants to identify the specific set of actionable changes up-regulating the translation efficiency of each WT Kozak sequence. We started from 230 haploinsufficient genes, from which we created a non-biased library of Kozak mutants. We obtained 5539 variants, 4838 of which are unique. As the destination vector, we used pWPT-EGFP-IRESmCherry. After assembly, the library of wild-type and variant Kozak sequences would substitute the EGFP Kozak sequence, directing EGFP expression. The obtained reporter bearing the library was used to transduce HEK293T cells.

Evaluation of Protein Levels from the Wild-Type and Variant Kozak Sequences

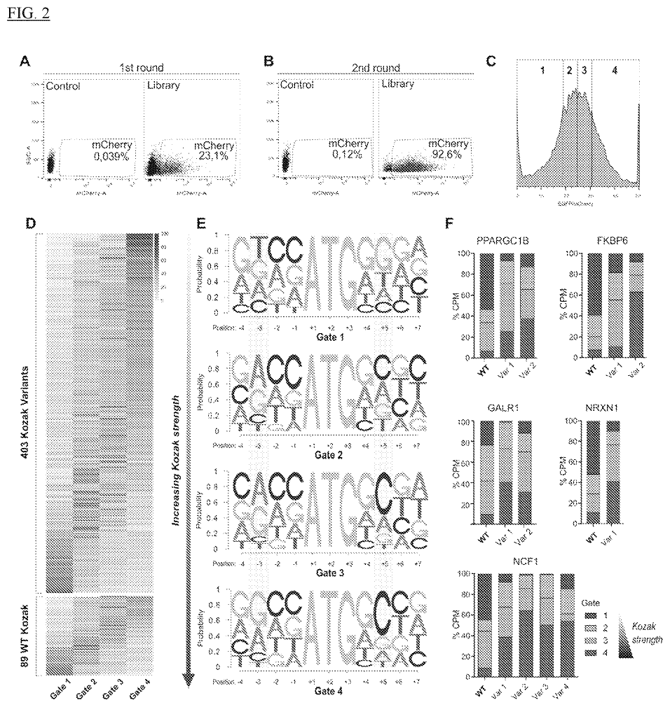

To quantify the translational efficiency of the wild-type and variant Kozak sequences of the HI genes, we cell sorted HEK293T cells transduced with the reporter bearing the library in four gates according to the normalised EGFP translational efficiency (EGFP/mCherry). In the first round, 5×106 mCherry positive cells were sorted to ensure 1000X library coverage (FIG. 2A). in the second round, the resulting mCherry positive cells were sorted according to their EGFP/mCherry ratio in 4 bins of different fluorescence intensity ratios (FIG. 2B, C). The Kozak sequence region from the cells collected in each bin was PCR-amplified. Deep sequencing of all fractions allowed us to compare the strength of each HI wildtype Kozak to its variants. 89 wild-type sequences and 403 variant sequences passed the statistical analysis (FIG. 2D). We then generated a motif for each of the four gates, representing the nucleotide frequency at each position of the Kozak sequence (FIG. 2E). Aiming at selecting Kozak variants up-regulating the corresponding WT, we selected only the variants with maximal distance from the respective WT. We obtained 47 wild-type and 149 variant sequences. From this list, we selected 5 HI genes and their corresponding variants for validation: PPARGC1B, FKBP6, GALR1, NRXN1, and NCF1 (FIG. 2F).

### Validation of Protein Up-Regulation by Selected Hit Kozak Sequence Variants

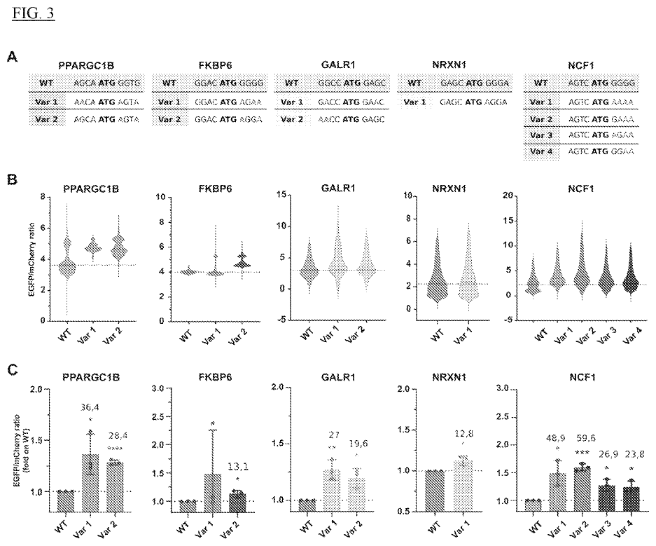

To validate the selected hits, we cloned each of the Kozak sequences (the wild-type and hit variants of the five selected genes) in place of the plasmid EGFP Kozak sequence in our reporter vector, creating one new plasmid for each sequence. We transiently transfected HEK293T cells with the respective wild-type and hit Kozak variants and measured the fluorescence by high content image analysis three days after transfection (FIG. 3). These analyses confirmed that 10 out of the 11 tested Kozak variants increase the translational efficiency compared to their respective wild-type sequence (FIG. 3B, C).

### Enhancement of NCF1 Translation by Base Editing of its Kozak Sequence

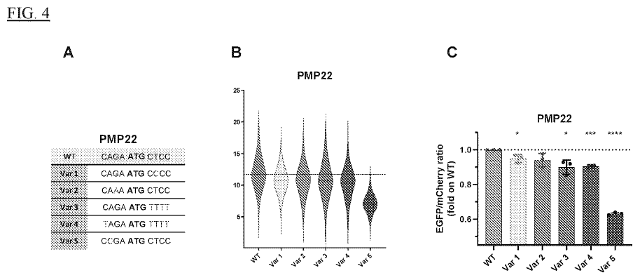

We then reproduced two variants that emerged from the screening (Var 2: SEQ ID NO:55; Var 4: SEQ ID NO:57) by base editing of the endogenous locus in Raji cells, a B lymphocyte cell line derived from Burkitt's lymphoma that constitutively expresses the gene of interest. We performed base editing by electroporating AncBE4max and the guide RNA sgNCF1 (SEQ ID NO:59) (FIG. 4). To improve the readout of the editing, we then decided to isolate cell clones, and we found and expanded clones having the desired base editor-mediated nucleotide changes equivalent to the Kozak NCF1 variant 2 and 4 (SEQ ID NO:55 and SEQ ID NO:57). Western blot analysis revealed increased expression of p47ₚₕₒₓ, the protein encoded by NCF1, with both variants compared to the wild-type (FIG. 4E, F). We also analysed the NCF1 mRNA level in the wild-type cells and the clones finding that they were unchanged. These results strongly support the idea that the increase in gene expression results from an enhancement in the translation of NCF1 due to the Kozak sequence editing (FIG. 4G). Sucrose gradient fractionation in the WT and edited cells showed that the increase in protein levels corresponds to the increased loading of mRNA on the polysomes (FIG. 4H, I). Collectively, these results showed that this is a new gene-editing approach targeting the Kozak sequence of a gene. It introduces through base editing suitable variants triggering the translational up-regulation of the target gene.

The following Table 1 shows the variant Kozak SEQ ID NO:1-58 of the invention.

The following Table 2 shows the gRNAs SEQ ID NO:59 and 60 of the invention.

The following Table 3 shows the variant Kozak SEQ ID NO:61-65 of the invention.

TABLE 1

| | | | | | ID | | |
|---|---|---|---|---|---|---|---|
| | | | OMIM | | wild type Kozak | | |
| Therapeutic | | | disease | wild-type | seqs - | Variant Kozak | SEQ ID |
| Area | Gene | Disease | # | Kozak seqs | map hg38 | seqs | NO |
| Developmental | DYRK1A | Intellectual | 614104 | GACGATGCATA | chr21: 37420371- | GACGATGCACA | 1 |
| disabilities | | developmental | | | 37420381 | GACGATGCGTA | 2 |
| | | disorder, | | | | GGCGATGCGTA | 3 |
| | | autosomal | | | | | |
| | | dominant 7 | | | | | |
| | GRIN2B | Intellectual | 613970 | GAAGATGAAGC | chr12: 13866193- | GAGGATGGAGC | 4 |
| | | developmental | | | 13866203 | GAGGATGAAGC | 5 |
| | | disorder, | | | | GGAGATGAGGC | 6 |
| | | automosomal | | | | GAAGATGGAGC | 7 |
| | | dominant 6. | | | | GGGGATGAAGC | 8 |
| | | with or | | | | | |
| | | without | | | | | |
| | | seizures | | | | | |
| | NRXN1 | Chromosome | 614332 | GAGCATGGGGA | chr2: 51028258- | GAGCATGAGGA | 9 |
| | | 2p16.3 | | | 51028268 | GAGCATGAAGA | 10 |
| | | deletion | | | | GAACATGGAAA | 11 |
| | | syndrome | | | | AAACATGAGGA | 12 |
| | | | | | | AAGCATGGAGA | 13 |
| | | | | | | GAGCATGGAAA | 14 |
| | STXBP1 | Developmental | 612164 | CGCCATGGCCC | chr9: 127612400- | CGTCATGGCCT | 15 |
| | | and epileptic | | | 127612410 | TGCCATGGCTT | 16 |
| | | encephalopathy | | | | CGCTATGGCCT | 17 |
| | | 4 | | | | TGTCATGGCCT | 18 |
| | | | | | | TGTTATGGCCT | 19 |
| | | | | | | CGTTATGGCCT | 20 |
| | | | | | | CGCTATGGTTT | 21 |
| | | | | | | CGCTATGGCCC | 22 |
| | | | | | | TGCCATGGCCT | 23 |
| | | | | | | TGCCATGGCTC | 24 |
| | | | | | | CGTCATGGCTC | 25 |
| | | | | | | CGTCATGGTCT | 26 |
| | | | | | | TGTTATGGCTC | 27 |
| | | | | | | CGCCATGGCTC | 28 |
| Metabolic | HNFIA | Maturity- | 600496 | AGCCATGGTTT | chr12: 120978765- | AGCCATGGCCC | 29 |
| syndrome | | onset diabetes | | | 120978775 | AGCCATGGCCT | 30 |
| | | of the young, | | | | AGCCATGGCTT | 31 |
| | | type 3 | | | | AACCATGGTTT | 32 |
| | | | | | | GGCCATGGTTT | 33 |
| | GHRL | Susceptibility | 601665 | GGCCATGCCCT | chr3: 10290165- | GGTCATGCCCT | 34 |
| | | to obesity | | | 10290175 | GGCTATGTCTT | 35 |
| | | | | | | GGTTATGTCCT | 36 |
| | | | | | | GGCCATGCCTT | 37 |
| | | | | | | GGCCATGTCTT | 38 |
| | PROX1 | Lymphatic | 601665 | AGTGATGCCTG | chr1: 213996532- | AGTGATGTCTG | 39 |
| | | vascular | | | 213996542 | AATGATGCCTG | 40 |
| | | defects, adult- | | | | AGTGATGCCTA | 41 |
| | | onset obesity | | | | | |
| Eye | EYA1 | Branchio-oto- | 113650 | GTCTATGGAAA | chr8: 71354890- | GTCTATGGAAG | 42 |
| disorder | | renal (BOR) | | | 71354900 | GTCTATGGAGA | 43 |
| | | syndrome | | | | | |
| | OPA1 | Optic atrophy | 165500 | CGGGATGTGGC | chr3: 193593374- | CGGAATGTAAC | 44 |
| | | | | | 193593384 | CAAGATGTAAC | 45 |
| | | | | | | CGGAATGTGGC | 46 |
| | | | | | | CAAGATGTGGC | 47 |
| | | | | | | TGGGATGTGGT | 48 |
| | | | | | | CGGGATGTGGT | 49 |
| | | | | | | CAGGATGTGGC | 50 |
| | COL2A1 | Stickler | 609508 | AGCCATGATTC | chr12: 48004306- | AGCCATGACCC | 51 |
| | | syndrome | | | 48004316 | AGCCATGACTC | 52 |
| | | type 1, | | | | AGTTATGATTT | 53 |
| | | nonsyndromic | | | | | |
| | | ocular | | | | | |
| Hematopoietic | NCF1 | Chronic | 233700 | AGTCATGGGGG | chr7: 74774028- | AGTCATGAAAA | 54 |
| disease | | granulomatous | | | 74774038 | AGTCATGGAAA | 55 |
| | | disease | | | | AGTCATGAGAA | 56 |
| | | (CGD) | | | | AGTCATGGGAA | 57 |
| | | | | | | AGTCATGGGAG | 58 |

TABLE 2

| | Target gene/ | | SEQ |
|---|---|---|---|
| gRNA | disease | Sequence | ID NO |
| sgNCF1 | NCF1/chronic | TGAAGGTGTCCCCCATGACT | 59 |
| | granulomatous | | |
| | disease | | |
| sgOPA1 | OPA1/optic | TCCCGCCGGCGGGGAGGTCA | 60 |
| | atrophy | | |

TABLE 3

| | | | | | ID | | |
|---|---|---|---|---|---|---|---|
| | | | OMIM | | wild type Kozak | | |
| Therapeutic | | | disease | wild-type | seqs - | Variant Kozak | SEQ ID |
| Area | Gene | Disease | # | Kozak seqs | map hg38 | seqs | NO |
| Motor | PMP22 | Charcot- | 118220 | CAGAATGCTCC | chr17:15260721- | CAGAATGCCCC | 61 |
| and | | Marie-Tooth | | | 15260731 | CAAAATGCTCC | 62 |
| sensory | | disease, type | | | | CAGAATGTTTT | 63 |
| neuropathy | | 1A | | | | TAGAATGTTTT | 64 |
| | | | | | | CCGAATGCTCC | 65 |

1. A variant Kozak nucleotide sequence obtained by genome editing methods acting as translational modulator of a protein-encoding gene in the treatment of a disease wherein the expression of said protein is altered, wherein said variant Kozak nucleotide sequence replaces, in vitro or in vivo, the wild-type Kozak sequence.

2. A variant Kozak nucleotide sequences according to claim 1, wherein said genome editing methods are selected from the group consisting of CRISPR-Cas homology-directed repair, CRISPR-Cas prime editing, CRISPR-Cas base editing or any other method based on programmable RNA-guided nuclease fused to effector proteins allowing for the introduction of one or more nucleotide conversions.

3. A variant Kozak nucleotide sequence according to claim 1 wherein said translation modulator is a translation enhancer or a translation repressor.

4. A variant Kozak nucleotide sequence according to claim 3 selected from the group consisting of SEQ ID NO:1-SEQ ID NO:58 as translation enhancers or selected from the group consisting of SEQ ID NO:61-SEQ ID NO:65 as translation repressors wherein said variant Kozak nucleotide sequences replace, in vitro or in vivo, the wild-type Kozak sequence.

5. A method of increasing the translational efficiency of a protein-encoding gene in the treatment of haploinsufficiency diseases comprising administering to a subject in need thereof a sufficient amount of the variant Kozak nucleotide sequence according to claim 4.

6. The method according to claim 5 wherein said haploinsufficiency disease is selected from the following disease classes: developmental disabilities, metabolic syndromes, eye disorders and hematopoietic diseases.

7. The method according to claim 6, wherein the developmental disability is selected from: intellectual developmental disorder, autosomal dominant 7; intellectual developmental disorder 6, with or without seizures; 2p16.3 deletion syndrome; developmental and epileptic encephalopathy 4.

8. The method according to claim 6, wherein:
when the developmental disability is intellectual developmental disorder, autosomal dominant 7, the nucleotide sequence is selected from SEQ ID NO:1 to SEQ ID NO:3;
when the developmental disability is intellectual developmental disorder, autosomal dominant 6, with or without seizures, the nucleotide sequence is selected from SEQ ID NO:4 to SEQ ID NO:8;
when the developmental disability is chromosome 2p16.3 deletion syndrome, the nucleotide sequence is selected from SEQ ID NO:9 to SEQ ID NO:14;
when the developmental disability is developmental and epileptic encephalopathy 4, the nucleotide sequence is selected from SEQ ID NO:15 to SEQ ID NO:28.

9. The method according to claim 6, wherein the metabolic syndrome is selected from: maturity-onset diabetes of the young, type 3; susceptibility to obesity; lymphatic vascular defects and/or adult-onset obesity.

10. The method according to claim 6, wherein:
when the metabolic syndrome is maturity-onset diabetes of the young, type 3, the nucleotide sequence is selected from SEQ ID NO:29 to SEQ ID NO:33;
when the metabolic syndrome is susceptibility to obesity, the nucleotide sequence is selected from SEQ ID NO;34 to SEQ ID NO:38;
when the metabolic syndrome is lymphatic vascular defects and/or adult-onset obesity, the nucleotide sequence is selected from SEQ ID NO:39 to SEQ ID NO:41.

11. The method according to claim 6, wherein the eye disorder is selected from branchiootorenal syndrome, optic atrophy, Stickler syndrome type 1, nonsyndromic ocular.

12. The method according to claim 6, wherein:
when the eye disorder is branchiootorenal syndrome, the nucleotide sequence is selected from SEQ ID NO:42 or SEQ ID NO:43;
when the eye disorder is optic atrophy, the nucleotide sequence is selected from SEQ ID NO:44 to SEQ ID NO:50;
when the eye disorder is Stickler syndrome type 1, nonsyndromic ocular, the nucleotide sequence is selected from SEQ ID NO:51 to SEQ ID NO:53.

13. The method according to claim 6 wherein when the hematopoietic disease is chronic granulomatous disease, said nucleotide sequence is selected from SEQ ID NO:54 to SEQ ID NO:58.

14. A gRNA designed to edit the wild-type Kozak sequence in order to obtain any one of the variant Kozak nucleotide sequences selected from the group SEQ ID NO:1-SEQ ID NO:58, characterized in that its targeting sequence corresponds to a target domain adjacent to a PAM sequence that is within 30 nucleotides to the ATG starting codon, either upstream or downstream.

15. A gRNA according to claim 14 editing the wild-type Kozak sequence of NCF1 wherein the gRNA has the nucleotide sequence SEQ ID NO:59.

16. A gRNA according to claim 14 editing the wild-type Kozak sequence of OPA1 wherein the gRNA has the nucleotide sequence SEQ ID NO:60.

17. Vector for genome editing comprising any one of the gRNAs according to claim 14.

18. Pharmaceutical composition comprising the vector according to claim 17, together with other suitable components for in vivo genome editing.

19. Pharmaceutical composition according to claim 18, which is intravenously injectable.

20. A method of treating haploinsufficiency diseases or gene duplication diseases comprising administering to a subject in need thereof a therapeutically sufficient amount of the variant Kozak nucleotide sequence according to claim 1.

21. The method according to claim 20, wherein the haploinsufficiency disease is selected from the group consisting of developmental disabilities, metabolic syndromes, eye disorders and hematopoietic diseases.

22. The method according to claim 21, wherein:
when the developmental disability is intellectual developmental disorder, autosomal dominant 7, the nucleotide sequence is selected from SEQ ID NO:1 to SEQ ID NO:3;
when the developmental disability is intellectual developmental disorder, autosomal dominant 6, with or without seizures, the nucleotide sequence is selected from SEQ ID NO:4 to SEQ ID NO:8;
when the developmental disability is chromosome 2p16.3 deletion syndrome, the nucleotide sequence is selected from SEQ ID NO:9 to SEQ ID NO:14;
when the developmental disability is developmental and epileptic encephalopathy 4, the nucleotide sequence is selected from SEQ ID NO:15 to SEQ ID NO:28.

23. The method according to claim 21, wherein:
when the metabolic syndrome is maturity-onset diabetes of the young, type 3, the nucleotide sequence is selected from SEQ ID NO:29 to SEQ ID NO:33;
when the metabolic syndrome is susceptibility to obesity, the nucleotide sequence is selected from SEQ ID NO; 34 to SEQ ID NO:38;
when the metabolic syndrome is lymphatic vascular defects and/or adult-onset obesity, the nucleotide sequence is selected from SEQ ID NO:39 to SEQ ID NO:41.

24. The method according to claim 21, wherein:
when the eye disorder is branchiootorenal syndrome, the nucleotide sequence is selected from SEQ ID NO:42 or SEQ ID NO:43;
when the eye disorder is optic atrophy, the nucleotide sequence is selected from SEQ ID NO:44 to SEQ ID NO:50;
when the eye disorder is Stickler syndrome type 1, nonsyndromic ocular, the nucleotide sequence is selected from SEQ ID NO:51 to SEQ ID NO:53.

25. The method according to claim 21, wherein when the hematopoietic disease is chronic granulomatous disease, said nucleotide sequence is selected from SEQ ID NO:54 to SEQ ID NO:58.

26. The method according to claim 20, wherein the gene duplication disease is selected from the group consisting of motor and sensory neuropathies.

27. The method according to claim 26, wherein the motor and sensory neuropathy is Charcot-Marie-Tooth disease type 1A, the nucleotide sequence is selected from SEQ ID NO:61 to SEQ ID NO:65.


SEQUENCE LISTING (74 sequences)

SEQ ID NO: 1 - DNA, 11 bp, Homo sapiens
  source 1..11: mol_type = other DNA
gacgatgcata

SEQ ID NO: 2 - DNA, 11 bp, synthetic construct
  misc_feature 1..11: note = Artificial
  source 1..11: mol_type = other DNA
gacgatgcaca

SEQ ID NO: 3 - DNA, 11 bp, synthetic construct
  misc_feature 1..11: note = Artificial
  source 1..11: mol_type = other DNA
gacgatgcgta

SEQ ID NO: 4 - DNA, 11 bp, synthetic construct
  misc_feature 1..11: note = Artificial
  source 1..11: mol_type = other DNA
ggcgatgcgta

SEQ ID NO: 5 - DNA, 11 bp, Homo sapiens
  misc_feature 1..11: note = Artificial
  source 1..11: mol_type = other DNA
gaggatggagc

SEQ ID NO: 6 - DNA, 11 bp, synthetic construct
  misc_feature 1..11: note = Artificial
  source 1..11: mol_type = other DNA
gaggatggagc

SEQ ID NO: 7 - DNA, 11 bp, synthetic construct
  misc_feature 1..11: note = Artificial
  source 1..11: mol_type = other DNA
gaggatgaagc

SEQ ID NO: 8 - DNA, 11 bp, synthetic construct
  misc_feature 1..11: note = Artificial
  source 1..11: mol_type = other DNA
ggagatgaggc

SEQ ID NO: 9 - DNA, 11 bp, synthetic construct
  misc_feature 1..11: note = Artificial
  source 1..11: mol_type = other DNA
gaagatggagc

SEQ ID NO: 10 - DNA, 11 bp, synthetic construct
  misc_feature 1..11: note = Artificial
  source 1..11: mol_type = other DNA
ggggatgaagc

SEQ ID NO: 11 - DNA, 11 bp, Homo sapiens
  source 1..11: mol_type = other DNA
gagcatgggga

SEQ ID NO: 12 - DNA, 11 bp, synthetic construct
  misc_feature 1..11: note = Artificial
  source 1..11: mol_type = other DNA
gagcatgagga

SEQ ID NO: 13 - DNA, 11 bp, synthetic construct
  misc_feature 1..11: note = Artificial
  source 1..11: mol_type = other DNA
gagcatgaaga

SEQ ID NO: 14 - DNA, 11 bp, synthetic construct
  misc_feature 1..11: note = Artificial
  source 1..11: mol_type = other DNA
gaacatggaaa

SEQ ID NO: 15 - DNA, 11 bp, synthetic construct
  misc_feature 1..11: note = Artificial
  source 1..11: mol_type = other DNA
aaacatgagga

SEQ ID NO: 16 - DNA, 11 bp, synthetic construct
  misc_feature 1..11: note = Artificial
  source 1..11: mol_type = other DNA
aagcatggaga

SEQ ID NO: 17 - DNA, 11 bp, synthetic construct
  misc_feature 1..11: note = Artifial
  source 1..11: mol_type = other DNA
gagcatggaaa

SEQ ID NO: 18 - DNA, 11 bp, Homo sapiens
  source 1..11: mol_type = other DNA
cgccatggccc

SEQ ID NO: 19 - DNA, 11 bp, synthetic construct
  misc_feature 1..11: note = Artificial
  source 1..11: mol_type = other DNA
cgtcatggcct

SEQ ID NO: 20 - DNA, 11 bp, synthetic construct
  misc_feature 1..11: note = Artificial
  source 1..11: mol_type = other DNA
tgccatggctt

SEQ ID NO: 21 - DNA, 11 bp, synthetic construct
  misc_feature 1..11: note = Artificial
  source 1..11: mol_type = other DNA
cgctatggcct

SEQ ID NO: 22 - DNA, 11 bp, synthetic construct
  misc_feature 1..11: note = Artificial
  source 1..11: mol_type = other DNA
tgtcatggcct

SEQ ID NO: 23 - DNA, 11 bp, synthetic construct
  misc_feature 1..11: note = Artificial
  source 1..11: mol_type = other DNA
tgttatggcct

SEQ ID NO: 24 - DNA, 11 bp, synthetic construct
  misc_feature 1..11: note = Artificial
  source 1..11: mol_type = other DNA
cgttatggcct

SEQ ID NO: 25 - DNA, 11 bp, synthetic construct
  misc_feature 1..11: note = Artificial
  source 1..11: mol_type = other DNA
cgctatggttt

SEQ ID NO: 26 - DNA, 11 bp, synthetic construct
  misc_feature 1..11: note = Artificial
  source 1..11: mol_type = other DNA
cgctatggccc

SEQ ID NO: 27 - DNA, 11 bp, synthetic construct
  misc_feature 1..11: note = Artificial
  source 1..11: mol_type = other DNA
tgccatggcct

SEQ ID NO: 28 - DNA, 11 bp, synthetic construct
  misc_feature 1..11: note = Artificial
  source 1..11: mol_type = other DNA
tgccatggctc

SEQ ID NO: 29 - DNA, 11 bp, synthetic construct
  misc_feature 1..11: note = Artificial
  source 1..11: mol_type = other DNA
cgtcatggctc

SEQ ID NO: 30 - DNA, 11 bp, synthetic construct
  misc_feature 1..11: note = Artificial
  source 1..11: mol_type = other DNA
cgtcatggtct

SEQ ID NO: 31 - DNA, 11 bp, synthetic construct
  misc_feature 1..11: note = Artificial
  source 1..11: mol_type = other DNA
tgttatggctc

SEQ ID NO: 32 - DNA, 11 bp, synthetic construct
  misc_feature 1..11: note = Artificial
  source 1..11: mol_type = other DNA
cgccatggctc

SEQ ID NO: 33 - DNA, 11 bp, Homo sapiens
  source 1..11: mol_type = other DNA
agccatggttt

SEQ ID NO: 34 - DNA, 11 bp, synthetic construct
  misc_feature 1..11: note = Artificial
  source 1..11: mol_type = other DNA
agccatggccc

SEQ ID NO: 35 - DNA, 11 bp, synthetic construct
  misc_feature 1..11: note = Artificial
  source 1..11: mol_type = other DNA
agccatggcct

SEQ ID NO: 36 - DNA, 11 bp, synthetic construct
  misc_feature 1..11: note = Artificial
  source 1..11: mol_type = other DNA
agccatggctt

SEQ ID NO: 37 - DNA, 11 bp, synthetic construct
  misc_feature 1..11: note = Artificial
  source 1..11: mol_type = other DNA
aaccatggttt

SEQ ID NO: 38 - DNA, 11 bp, synthetic construct
  misc_feature 1..11: note = Artificial
  source 1..11: mol_type = other DNA
ggccatggttt

SEQ ID NO: 39 - DNA, 11 bp, Homo sapiens
  source 1..11: mol_type = other DNA
agccatggttt

SEQ ID NO: 40 - DNA, 11 bp, synthetic construct
  misc_feature 1..11: note = Artificial
  source 1..11: mol_type = other DNA
ggtcatgccct

SEQ ID NO: 41 - DNA, 11 bp, synthetic construct
  misc_feature 1..11: note = Artificial
  source 1..11: mol_type = other DNA
ggctatgtctt

SEQ ID NO: 42 - DNA, 11 bp, synthetic construct
  misc_feature 1..11: note = Artificial
  source 1..11: mol_type = other DNA
ggttatgtcct

SEQ ID NO: 43 - DNA, 11 bp, synthetic construct
  misc_feature 1..11: note = Artificial
  source 1..11: mol_type = other DNA
ggccatgcctt

SEQ ID NO: 44 - DNA, 11 bp, synthetic construct
  misc_feature 1..11: note = Artificial
  source 1..11: mol_type = other DNA
ggccatgtctt

SEQ ID NO: 45 - DNA, 11 bp, Homo sapiens
  source 1..11: mol_type = other DNA
agtgatgcctg

SEQ ID NO: 46 - DNA, 11 bp, synthetic construct
  misc_feature 1..11: note = Artificial
  source 1..11: mol_type = other DNA
agtgatgtctg

SEQ ID NO: 47 - DNA, 11 bp, synthetic construct
  misc_feature 1..11: note = Artificial
  source 1..11: mol_type = other DNA
aatgatgcctg

SEQ ID NO: 48 - DNA, 11 bp, synthetic construct
  misc_feature 1..11: note = Artificial
  source 1..11: mol_type = other DNA
agtgatgccta

SEQ ID NO: 49 - DNA, 11 bp, Homo sapiens
  source 1..11: mol_type = other DNA
gtctatggaaa

SEQ ID NO: 50 - DNA, 11 bp, synthetic construct
  misc_feature 1..11: note = Artificial
  source 1..11: mol_type = other DNA
gtctatggaag

SEQ ID NO: 51 - DNA, 11 bp, synthetic construct
  misc_feature 1..11: note = Artificial
  source 1..11: mol_type = other DNA
gtctatggaga

SEQ ID NO: 52 - DNA, 11 bp, Homo sapiens
  source 1..11: mol_type = other DNA
cgggatgtggc

SEQ ID NO: 53 - DNA, 11 bp, synthetic construct
  misc_feature 1..11: note = Artificial
  source 1..11: mol_type = other DNA
cggaatgtaac

SEQ ID NO: 54 - DNA, 11 bp, synthetic construct
  misc_feature 1..11: note = Artificial
  source 1..11: mol_type = other DNA
caagatgtaac

SEQ ID NO: 55 - DNA, 11 bp, synthetic construct
  misc_feature 1..11: note = Artificial
  source 1..11: mol_type = other DNA
cggaatgtggc

SEQ ID NO: 56 - DNA, 11 bp, synthetic construct
  misc_feature 1..11: note = Artificial
  source 1..11: mol_type = other DNA
caagatgtggc

SEQ ID NO: 57 - DNA, 11 bp, synthetic construct
  misc_feature 1..11: note = Artificial
  source 1..11: mol_type = other DNA
tgggatgtggt

SEQ ID NO: 58 - DNA, 11 bp, synthetic construct
  misc_feature 1..11: note = Artificial
  source 1..11: mol_type = other DNA
cgggatgtggt

SEQ ID NO: 59 - DNA, 11 bp, synthetic construct
  misc_feature 1..11: note = Artificial
  source 1..11: mol_type = other DNA
caggatgtggc

SEQ ID NO: 60 - DNA, 11 bp, Homo sapiens
  source 1..11: mol_type = other DNA
agccatgattc

SEQ ID NO: 61 - DNA, 11 bp, synthetic construct
  misc_feature 1..11: note = Artificial
  source 1..11: mol_type = other DNA
agccatgaccc

SEQ ID NO: 62 - DNA, 11 bp, synthetic construct
  misc_feature 1..11: note = Artificial
  source 1..11: mol_type = other DNA
agccatgactc

SEQ ID NO: 63 - DNA, 11 bp, synthetic construct
  misc_feature 1..11: note = Artificial
  source 1..11: mol_type = other DNA
agttatgattt

SEQ ID NO: 64 - DNA, 11 bp, Homo sapiens
  source 1..11: mol_type = other DNA
agtcatggggg

SEQ ID NO: 65 - DNA, 11 bp, synthetic construct
  misc_feature 1..11: note = Artificial
  source 1..11: mol_type = other DNA
agtcatgaaaa

SEQ ID NO: 66 - DNA, 11 bp, synthetic construct
  misc_feature 1..11: note = Artificial
  source 1..11: mol_type = other DNA
agtcatgggaa

SEQ ID NO: 67 - DNA, 11 bp, synthetic construct
  misc_feature 1..11: note = Artificial
  source 1..11: mol_type = other DNA
agtcatgagaa

SEQ ID NO: 68 - DNA, 11 bp, synthetic construct
  misc_feature 1..11: note = Artificial
  source 1..11: mol_type = other DNA
agtcatggaaa

SEQ ID NO: 69 - DNA, 11 bp, synthetic construct
  misc_feature 1..11: note = Artificial
  source 1..11: mol_type = other DNA
agtcatgggag

SEQ ID NO: 70 - DNA, 11 bp, synthetic construct
  source 1..11: mol_type = other DNA
cagaatgcccc

SEQ ID NO: 71 - DNA, 11 bp, synthetic construct
  source 1..11: mol_type = other DNA
caaaatgctcc

SEQ ID NO: 72 - DNA, 11 bp, synthetic construct
  source 1..11: mol_type = other DNA
cagaatgtttt

SEQ ID NO: 73 - DNA, 11 bp, synthetic construct
  source 1..11: mol_type = other DNA
tagaatgtttt

SEQ ID NO: 74 - DNA, 11 bp, synthetic construct
  source 1..11: mol_type = other DNA
ccgaatgctcc